(19) United States
(12) Patent Application Publication
(10) Pub. No.: US 2026/0224231 A1
(43) Pub. Date: Aug. 6, 2026

(54) LIGHTWEIGHT ONE PIECE CAN ACTUATOR FOR THE DELIVERY OF MICROPROJECTION ARRAY PATCHES (MAP)

(71) Applicant: VAXXAS PTY LIMITED, Queensland (AU)

(72) Inventors: Talia ROSE, Queensland (AU); Romulo DE MACEDO, Queensland (AU); Douglas MAIR, Queensland (AU); Scott FRY, Queensland (AU); HONG SENG LEE, Queensland (AU); Steven CARTER, Queensland (AU)

(21) Appl. No.: 19/153,236

(22) PCT Filed: Feb. 5, 2024
(86) PCT No.: PCT/AU2024/050067

§ 371 Date: Aug. 1, 2025

Related U.S. Application Data
(60) Provisional application No. 63/443,385, filed on Feb. 4, 2023.

(51) Int. Cl. A61B 17/20; A61M 37/00

(52) U.S. Cl. CPC A61B 17/205; A61M 37/0015; A61M 2037/0023; A61M 2037/0053; A61M 2209/06

(57) ABSTRACT

The present invention relates to improved applicators and methods of manufacturing applicators for administering microprojection arrays to skin and methods of administering microprojection arrays. In particular, the present invention relates to compact, stable, one-piece can, self-contained mechanical energy storage for delivery of a microprojection array to the skin.

### FIELD OF THE INVENTION

The present invention relates to improved applicators and methods of manufacturing applicators for administering microprojection arrays to skin and methods of administering microprojection arrays. In particular, the present invention relates to compact, stable, one-piece can, self-contained mechanical energy storage for delivery of a microprojection array to the skin.

### BACKGROUND OF THE INVENTION

Recently, new methods of delivering drugs and other bioactive materials have been developed that are more convenient, provide superior efficacy or enhanced performance compared to intramuscular and intradermal injection. Intradermal injection is limited by cross-contamination through needle-stick injuries in health workers, injection phobia from a needle and syringe, and the inability for needle and syringe methodology to target key cells in the outer skin layers. There still exists a need for a lightweight and compact single use applicator, easily manufactured, that can be triggered by the user without discomfort to the user or patient, and/or enable the patient to use the applicator by self-administration, and/or target the more challenging geriatric and/or paediatric populations. The delivery of a microprojection array patch (MAP) with minimal user trigger force and pressure on the patient, is highly desirable.

US Patent Publication No. 2009/0198189 describes a device for applying a microneedle array to a skin surface in which the device is comprised of a base which defines a skin contacting plane, a microneedle array and a connecting member having a portion affixed to the base through a hinge and another portion affixed to the microneedle array.

US Patent Publication No. 2011/0276027 also describes an applicator for microneedles in which the applicator comprises an energy-storing element which upon application of force cause the compressed element to extend or transition from a first to a second configuration releasing the stored energy to deploy a member which is configured to hold a microneedle array.

U.S. Pat. No. 8,540,672 describes an applicator including a housing, a slidably disposed applicator plate, and a compression spring. The applicator plate is moveable between a retracted position and a deployed position and has an engaging surface suitable for mashing up against a microneedle patch and pressing it against a skin surface. A docking system transfers the microneedle patch from a support to the applicator without requiring a user to handle the microneedle patch directly. Once mounted in the applicator, the microneedle patch is deployed against a skin surface of a patient for delivery of a desired agent via a microneedle array contained on the patch.

US Patent Publication No. 2008/0009811 describes an applicator capable of sensing a controlled distance from a skin surface and propelling a microneedle array across this distance and into the skin surface is disclosed. A method of applying a microneedle array to a skin surface by placing the microneedle array a predetermined distance away from the skin surface and propelling the microneedle array into the skin surface is disclosed.

WO 2014/058746 describes an applicator for applying a microneedle device to a skin surface. The applicator can include a microneedle device, a housing, and a connecting member. The connecting member can be configured to allow the microneedle device to move between: (i) a first position in which at least a portion of the microneedle device extends beyond the housing; and (ii) a second position in which the microneedle device is recessed within the housing when a threshold application force is applied to the microneedle device in a direction substantially perpendicular with respect to the microneedle device.

U.S. Pat. No. 11,147,954 describes an applicator device having a housing having an upper and lower portion and having an internal face and an external face wherein the external face has a flexible section that when collapsed actuates the device and a cantilevered ring where the microprojection array is directly contacted by the cantilevered ring when the cantilevered ring is activated and where the microprojection array is releasably detached from the device after the microprojection array is contacted by the cantilevered ring,

U.S. Pat. No. 11,464,957 describes a device for applying a microprojection array to the skin of a mammal, the device having a housing which comprises a top shell having a collapsible trigger operably linked to a pre-loaded dome, and a bottom shell and a spring holding the microprojection array, wherein the pre-loaded dome is encased in the housing such that when the trigger is collapsed the dome transitions from a loaded position to an unloaded position, thereby contacting the spring and propelling the microprojection array through a space between the device and the mammal's skin and into the mammal's skin.

Despite the development of numerous devices for the application of microprojection and microneedle arrays there remain difficulties in devising a device and method for the arrays to overcome the natural elasticity of the skin and penetrating the skin to deliver the required drug dosage while maintaining comfort and ease of use for the patient. Prior art applicators are also prone to permit moisture ingress into the device thereby de-stabilizing the drug to be delivered. Current applicators have several moving parts and are not constructed as a single unit, but need to be assembled from several components. The present invention provides devices and methods for projecting high density microprojection arrays (e.g. microprojection arrays having more than 1,000projections/cm².) into the skin to deliver the required drug dosages.

In view of the above, it would be desirable to provide for a lightweight and compact single use applicator that can be triggered easily by the user without discomfort to the patient. It would be also desirable to provide applicators that enable the patient to use the applicator by self-administration or by administration by a second party. Furthermore, it would be desirable to provide a device that is simple to assemble, has fewer moving parts and significantly reduces the ingress of moisture so that the drug to be delivered is stable for longer time periods. It would also be desirable to enable the delivery of high density microprojection arrays at high speed with minimal user trigger force and pressure on the patient. The ease of administration, the reduction in patient discomfort, and the superior delivery of drugs and vaccines are highly desirable for the sake of public health.

The present invention seeks to provide for one or more of the desirable outcomes outlined above, or to at least provide a useful alternative to prior art solutions.

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

### SUMMARY OF THE PRESENT INVENTION

The present invention relates to compact, stable, self-contained mechanical energy storage devices for the administration of a microprojection array. The mechanisms and applicators of the present invention provide for high-speed actuation of a microprojection array patch (MAP) while requiring a low trigger force from the user. Such mechanisms may be achieved by putting a high performance asymmetric bi-stable metal dome which is close to the dome's critical snap-through state and stabilizing the dome in the device with a retaining ring which does not encase the dome either in the loaded state or while it is transiting from an unloaded to loaded state.

Metal discs or strips stamped in the shape of a dome or a strip of metal can exhibit bi-stable positions when specifically designed with pre-determined parameters with respect to stamping profile, height, thickness and steel properties. When a static or dynamic load is exerted on the dome, the dome will start buckling until a critical load is reached. Once the critical load is reached the dome suddenly accelerates and inverts its geometry (“snap through”) without further loading.

An asymmetric bi-stable dome may be designed such that the force to load the dome in its energized state is higher than the force required for triggering the dome to return to its unloaded position. This asymmetry means the dome stores potential mechanical energy, which can be released in a highly transient timeframe due to a lower energy trigger.

The present invention relates to microprojection array applicators comprising such domes that provide application of microprojection arrays to the skin for the delivery of substances, in particular vaccines. The dome devices of the present invention are particularly useful for applying small area, high-density microprojection arrays having densely packed microprojections. In addition, the microprojection array applicators of the present invention are useful in the application of microprojection arrays that are of low mass and which may be projected into the skin by transiting a space between the applicator and the skin. In other words, the device and methods of the present invention provide applicators in which the low mass microprojection array is propelled through space prior to penetrating the skin.

The present invention also relates to methods of using the microprojection array applicators for applying arrays to the skin of a subject. The present invention provides a compact mechanism which enables the design of high density microprojection array applicators, able to provide high-velocities for low trigger forces and low impact on the patient, while containing its stored energy for an extended time.

The devices of the present invention can be used as a mechanical potential energy storage unit and actuator for a microprojection array. In this application the patch is accelerated or struck at high speed by the transiting dome and propelled toward the patient skin. The attained velocity enables the patch to counter the natural elasticity of the skin and pierce the skin, and ultimately deliver the compounds coated on the microprojections of the array and into the skin tissues. Strain rate plus kinetic energy combine to rupture the stratum corneum and drive the microprojections to a required depth.

This mechanical potential energy storage unit and actuator (i.e. dome system) interfaces with the inner mechanism of the applicator (i.e. patch attach inner mechanism) which enables the assembly of the coated patch and its triggering upon contact with the transiting dome. The system provides guidance to the microprojection array while accelerating the array.

The present invention relates to devices for delivering a microprojection array into the skin of a mammal comprising a primed dome; a retaining ring; a main body; a can; a microprojection array; and a foil seal and optionally a desiccant ring.

The present invention relates to devices which are self-contained units and where the components are contained within the can and the device has no necking or sealing on the exterior of the can except for the foil seal which is adhered to the base of the can.

The present invention relates to devices where the microprojection array comprises a base having a plurality of microprojections and a spigot molded to the base and where the spigot attaches to the main body.

The present invention relates to devices where the dome has a flattened outer edge.

The present invention relates to devices where the flattened outer edge is from 3.3 to 3.6 mm.

The present invention relates to devices where the retainer ring continuously presses on the flattened outer edge of the dome.

The present invention relates to devices where the can is made of aluminum.

The present invention relates to devices where the aluminum is from 90 to 180 μm thick.

The present invention relates to devices where the aluminum is from 90 to 120 μm thick.

The present invention relates to devices where the device also contains a desiccant.

The present invention relates to devices where the desiccant is included inside the device.

The present invention relates to devices where the desiccant is included in the foil seal.

The present invention relates to devices where the desiccant is contained within the main body.

The present invention relates to devices where the microprojection array has from about 1000 to 3000 microprojections.

The present invention relates to devices for delivering a microprojection array into the skin of a mammal having a primed dome seated within a main body which is held in place by a retaining ring, where the microprojection array is held by the main body and the main body is contained within a can which has a single opening, and where a foil seal is attached to the can opening.

The present invention relates to methods for assembling a device for delivering a microprojection array into the skin of a mammal comprising: locating a primed dome onto a seat within a main body of the device; press fitting a retaining ring to the main body of the device thereby securing the dome in place; inserting the main body of the device into a can and heat pressing the can so that the main body is adhered to the can; inserting the microprojection array into the main body such that one or more retention features of the main body hold the microprojection array in place; and heat pressing a foil seal to the base of the can.

Broad forms of the invention and their respective features can be used in conjunction, interchangeably and/or independently, and reference to separate broad forms is not intended to be limiting.

### BRIEF DESCRIPTION OF THE DRAWINGS

Various examples and embodiments of the present invention will now be described with reference to the accompanying drawings, in which:

FIG. 1 is an exploded view drawing of one embodiment of a device of the present invention which includes the can, retaining ring, dome, main body, MAP and foil covering.

FIG. 2 is a cross-sectional representation of the main body of the present invention.

FIG. 3 is a schematic drawing of one embodiment of the main body of the present invention shown from the top.

FIG. 4 is a schematic drawing of one embodiment of the main body of the present invention shown from the bottom.

FIG. 5 is a schematic drawing of one embodiment of the main body of the present invention shown from an angle from the top.

FIG. 6 is a schematic drawing of one embodiment of the main body of the present invention shown from an angle from the bottom.

FIG. 7 is a schematic cross-sectional drawing of one embodiment of the main body of the present invention with the MAP inserted into the main body.

FIG. 8A is a drawing of one embodiment of the can; and FIG. 8B is a drawing of the can into which the main body and MAP are placed.

FIGS. 9A to 9E are drawings of successive steps in constructing the applicator device.

FIG. 10 is a drawing of a MAP with a spigot.

FIG. 11A is a schematic cross-sectional drawing of the assembled device; FIG. 11B is a cross-sectional schematic of the insertion of the dome in the main body secured by the retaining ring; FIG. 11C is a cross-sectional schematic of the securing of the spigot to the main body; and FIG. 11D is a cross-sectional schematic of the attachment of the main body to the can with the foil overlay.

FIG. 12A is a schematic drawing of a top view of one embodiment of the desiccant ring; FIG. 12B is a schematic cross-sectional drawing of the desiccant ring; and FIG. 12C is a schematic drawing of an angled view of the desiccant ring.

FIG. 13A is a schematic cross-sectional drawing of the spigot in combination with the microprojection array (MAP); FIG. 13B is a schematic cross-sectional drawing of the top portion of the spigot; FIG. 13C is a schematic cross-sectional drawing of the lower portion of the spigot; FIG. 13D is a schematic cross-sectional drawing of the MAP; and FIG. 13E is a schematic drawing of the top portion of the MAP.

FIG. 14 is a schematic drawing of a top view of the foil tab.

FIG. 15A is schematic cross-sectional drawing of one embodiment of the main body of the device; FIG. 15B is a schematic cross-sectional drawing of how the main body and retaining ring are joined; FIG. 15C is schematic cross-sectional drawing of the detailed structure of the portion of the main body which holds the MAP; FIG. 15D is schematic drawing of the top of the main body of the device; and FIG. 15E is detailed schematic drawing of the main body of the device.

FIG. 16A is a schematic drawing of the top of the retaining ring; FIG. 16B is a schematic cross-sectional drawing of the retaining ring; and FIG. 16C is a magnified portion of the schematic drawing in 16B that shows the details of the exterior surface of the retaining ring.

FIG. 17 is a schematic drawing of the top of the can; FIG. 17B is a schematic cross-sectional drawing of the can; and FIG. 17C is a magnified schematic cross-sectional drawing of the interface between the can and the main body of the device.

FIG. 18A is a schematic cross-sectional drawing of the device without the spigot/MAP inserted; and FIG. 18A is a schematic cross-sectional drawing of the device with the spigot/MAP inserted.

FIG. 19 is a representation of the outside of the device.

FIG. 20 is a schematic cross-sectional drawing of one embodiment of a stepped microprojection.

### DETAILED DESCRIPTION OF THE PREFERRED EMBODIMENTS

The present invention relates to compact, stable, self-contained mechanical energy storage devices for the administration of a microprojection array (MAP). The mechanisms and applicators of the present invention provide for actuation of the MAP at high speeds (e.g. 18-28 m/s) using a microprojection array patch (MAP) with low mass (e.g. around 550 mg) while requiring a low trigger force (e.g. 15-50 N) from the user.

High performance asymmetric bi-stable domes which provide high speeds in the 18-24 m/s range have a loading force in the range of 200-300 Newtons and a trigger force around 100 Newtons (i.e. an approximate weight of 10 kg at standard gravity acceleration). Trigger force may result in a discomfort both for the user who needs to provide the large device trigger force and the patient who feels the large device pressure on the skin when triggering.

The devices and methods of the present invention provide a mechanism to lower the force when triggering the application device to a comfortable range of approximately 15-50 Newtons, while preserving or increasing the dome's velocity, such that the dome may accelerate a 300-600 mg projectile (such as a microprojection array) to a velocity of approximately 18-28 m/s. The domes of the present invention are secured using a dome retention ring.

The devices of the present invention must have the dome correctly integrated into the device, so that the energy release generated by triggering the dome is transferred into the MAP. A dome without the dome retention ring will “jump” in the device and the acceleration of the patch will be adversely impacted. An efficient coupling of the dome to the patch results in efficient acceleration of the patch and a successful application of the patch to the skin.

In one embodiment the dome may be made from a hardened stainless steel strip laser cut in an approximately 31.1 mm diameter disc, with a centred, approximately 3.0 mm diameter hole. Other embodiments of the domes include diameters which range from about 5 to 80 mm, or from about 5 to 70 mm or from about 5 to 60 mm or from about 5 to 50 mm or from about 5 to 40 mm or from about 5 to 30 mm or from about 5 to about 20 mm or from about 10 to 80 mm, or from about 10 to 70 mm or from about 10 to 60 mm or from about 10 to 50 mm or from about 10 to 40 mm or from about 10 to 30 mm or from about 10 to about 20 mm or from about 20 to 80 mm, or from about 20 to 70 mm or from about 20 to 60 mm or from about 20 to 50 mm or from about 20 to 40 mm or from about 20 to 30 mm or from about 30 to 80 mm, or from about 30 to 70 mm or from about 30 to 60 mm or from about 30 to 50 mm or from about 30 to 40 mm or from about 40 to 80 mm, or from about 40 to 70 mm or from about 40 to 60 mm or from about 40 to 50 mm or from about 50 to 80 mm, or from about 50 to 70 mm or from about 50 to 60 mm. The thickness of the dome may be from about 0.1 to 2 mm or from about 0.1 to 1.5 mm or from about 0.1 to 1.0 mm or from about 0.1 to 0.5 mm or from about 0.25 to 2.0 mm or from about 0.25 to 1.5 mm or from about 0.25 to 1.0 mm or from about 0.25 to 0.5 mm or from about 0.5 to 2 mm or from about 0.5 to 1.5 mm or from about 0.5 to 1.0 mm or from about 0.75 to 2.0 mm or from about 0.75 to 1.5 mm or from about 0.75 to 1.0 mm or from about 1.0 to 2.0 mm or from about 1.0 to 1.5 mm or from about 1.5 to 2.0 mm. The hole diameter in the dome may be from about 0% to 70% of the dome or from about 0% to 60% of the dome or from about 0% to 50% of the dome or from about 0% to 40% of the dome or from about 0% to 30% of the dome or from about 0% to 20% of the dome or from about 0% to 10% of the dome or from about 10% to 70% of the dome or from about 10% to 60% of the dome or from about 10% to 50% of the dome or from about 10% to 40% of the dome or from about 10% to 30% of the dome or from about 10% to 20% of the dome or from about 20% to 70% of the dome or from about 20% to 60% of the dome or from about 20% to 50% of the dome or from about 20% to 40% of the dome or from about 20% to 30% of the dome or from about 30% to 70% of the dome or from about 30% to 60% of the dome or from about 30% to 50% of the dome or from about 30% to 40% of the dome or from about 40% to 70% of the dome or from about 40% to 60% of the dome or from about 40% to 50% of the dome or from about 50% to 70% of the dome or from about 50% to 60%. The yield strength of the dome may be from about 400 to 3500 MPa, or from about 400 to 3000 MPa, or from about 400 to 2500 MPa or from about 400 to 2000 MPa, or from about 400 to 1500 MPa, or from about 400 to 1000 MPa, or from about 400 to 500 MPa, or from about 1000 to 3500 MPa or from about 1000 to 3000 MPa, or from about 1000 to 2500 MPa or from about 1000 to 2000 MPa, or from about 1000 to 1500 MPa or from about 1500 to 3500 MPa, or from about 1500 to 3000 MPa, or from about 1500 to 2500 MPa, or from about 1500 to 2000 MPa, or from about 2000 to 3500 MPa or from about 2000 to 3000 MPa or from about 2000 to 2500 MPa or from about 2500 to about 3500 MPa, or from about 2500 to about 3000 MPa. The tensile strength of the dome may be from about 250 to 2400 MPa, or from about 250 to 2000 MPa, or from about 250 to 1500 MPa or from about 250 to 1000 MPa, or from about 250 to 500 MPa, or from about 500 to 2400 MPa, or from about 500 to 2000 MPa, or from about 500 to 1500 MPa or from about 500 to 1000 MPa, or from about 750 to 2400 MPa or from about 750 to 2000 MPa, or from about 750 to 1500 MPa or from about 750 to 1000 MPa, or from about 1000 to 2400 MPa, or from about 1000 to 2000 MPa, or from about 1000 to 1500 MPa, or from about 1500 to 2400 MPa or from about 1500 to 200 MPa.

The device contains a high performance asymmetric bi-stable dome spring with a circumferentially flat lip which is 3.0-3.6 mm wide and a domed central region, with an approximately 3 mm centre hole at the apex of the central region. The transition radius between the lip and the dome central region is a fold line. A natural slight second curvature appears in the dome due to the anisotropy induced by the grain structure of the steel.

The central region of the dome may be “loaded” by displacing it perpendicularly to the flat of the dome's base until the concavity inverts through buckling (“snap-through”).

The dome may be considered a shell structure (a three-dimensional solid whose thickness is very small compared with its other dimensions). When a compressive load is applied axially to the dome, its geometry evolves (i.e. deformation) under the increasing bending moment while accommodating the build-up of membrane and shear forces, and related stresses. After reaching a defined load some areas of the dome start to experience buckling, meaning that locally these areas become unstable and are poised to snap-through to minimise their energy level. However, more areas of the dome are still in the elastic behaviour (and would return to the initial geometry if the load were removed), than there are areas of buckling. As the forced deformation increases, more and more of local areas of the dome are buckling which results in reducing the load experienced by the dome, until a peak load, and consequent decrease of load. Ultimately there comes a point where the resultant buckling of the components becomes similar to the elastic back-force resultant of the non-buckling component, which results in a critically unstable dome. Any further deformation, vibration, stress etc. makes the domes enter a highly transient behaviour where the buckling propagates to the full surface of the dome, resulting in the dynamic inversion of the dome. The dome inverts in order to minimise the bending moment, shear and membrane stresses, and reaches a lower energetic state (the inverted state). The transient nature of the inversion results in a high acceleration and deceleration of the centre part of the dome (apex), which can be used as a high-speed actuator to project a device such as a microprojection array.

In order to trigger the dome, the user needs to bring the dome to this critical state where the buckling propagates to the full dome.

The trigger force needs to be tailored to fall in a range, where the maximum corresponds to a force which is considered too high to deliver by a user and/or to be received by a patient, and the minimum corresponds to a force which is sufficient to prevent any unintentional triggering. The critical force can vary with imperfections in the dome (stamping, grain, defects, dints etc.), with the triggering (off-centring, angle, shape and size), with the dynamic of the triggering (low speed, high impact speed, vibrations) and stress variation (temperature, humidity, dilatation of steel/plastic). Therefore, some buffering needs to be considered in choosing the ends of the trigger force range. The range of the triggering force for the dome may be from 5 to 100 N, or from 5 to 90 N or from 5 to 80 N, or from 5 to 70 N or from 5 to 60 N, or from 5 to 50 N or from 5 to 40 N, or from 5 to 30 N or from 5 to 20 N or from 5 to 10 N, or from 10 to 100 N, or from 10 to 90 N or from 10 to 80 N, or from 10 to 70 N or from 10 to 60 N, or from 10 to 50 N or from 10 to 40 N, or from 10 to 30 N or from 10 to 20 N, or from 20 to 100 N or from 20 to 90 N or from 20 to 80 N, or from 20 to 70 N or from 20 to 60 N, or from 20 to 50 N or from 20 to 40 N, or from 20 to 30 N or from 30 to 100 N or from 30 to 90 N or from 30 to 80 N, or from 30 to 70 N or from 30 to 60 N, or from 30 to 50 N or from 30 to 40 N, or from 40 to 100 N or from 40 to 90 N or from 40 to 80 N, or from 40 to 70 N or from 40 to 60 N, or from 40 to 50 N or from 50 to 200 N, or from 50 to 90 N or from 50 to 80 N, or from 50 to 70 N or from 50 to 60 N, or from 60 to 100 N or from 60 to 90 N or from 60 to 80 N, or from 60 to 70 N or from 70 to 100 N or from 70 to 90 N, or from 70 to 80 N or from 80 to 100 N or from 80 to 90 N, or from 90 to 100 N. The range of the triggering force for an stand-alone dome may be from 100 to 200 N, or from 100 to 190 N or from 100 to 180 N, or from 100 to 170 N or from 100 to 160 N, or from 100 to 150 N or from 100 to 140 N, or from 100 to 130 N or from 100 to 120 N, or from 100 to 110 N or from 110 to 200 N, or from 110 to 190 N or from 110 to 180 N, or from 110 to 170 N or from 110 to 160 N, or from 110 to 150 N or from 110 to 140 N, or from 110 to 130 N or from 110 to 120 N or from 120 to 200 N, or from 120 to 190 N or from 120 to 180 N, or from 120 to 170 N or from 120 to 160 N, or from 120 to 150 N or from 120 to 140 N, or from 120 to 130 N or from 130 to 200 N, or from 130 to 190 N or from 130 to 180 N, or from 130 to 170 N or from 130 to 160 N, or from 130 to 150 N or from 130 to 140 N, or from 140 to 200 N, or from 140 to 190 N or from 140 to 180 N, or from 140 to 170 N or from 140 to 160 N, or from 140 to 150 N or from 150 to 200 N, or from 150 to 190 N or from 150 to 180 N, or from 150 to 170 N or from 150 to 160 N, or from 170 to 200 N or from 170 to 200 N, or from 170 to 190 N or from 170 to 180 N, or from 180 to 200 N or from 180 to 190 N, or from 190 to 200 N.

The range of the loading force for the dome may be from 100 to 400 N, or from 100 to 350 N or from 100 to 300 N, or from 100 to 250 N or from 100 to 200 N, or from 100 to 200 N or from 100 to 150 N, or from 150 to 400 N or from 150 to 350 N, or from 150 to 300 N or from 150 to 250 N or from 150 to 200 N, or from 200 to 400 N or from 250 to 350 N, or from 200 to 300 N or from 200 to 250 N, or from 250 to 400 N or from 250 to 350 N or from 250 to 300 N or from 300 to 400 N, or from 300 to 350 N or from 350 to 400 N. The range of the loading force for an stand-alone dome may be from 100 to 200 N, or from 100 to 190 N or from 100 to 180 N, or from 100 to 170 N or from 100 to 160 N, or from 100 to 150 N or from 100 to 140 N, or from 100 to 130 N or from 100 to 120 N, or from 100 to 110 N or from 110 to 200 N, or from 110 to 190 N or from 110 to 180 N, or from 110 to 170 N or from 110 to 160 N, or from 110 to 150 N or from 110 to 140 N, or from 110 to 130 N or from 110 to 120 N or from 120 to 200 N, or from 120 to 190 N or from 120 to 180 N, or from 120 to 170 N or from 120 to 160 N, or from 120 to 150 N or from 120 to 140 N, or from 120 to 130 N or from 130 to 200 N, or from 130 to 190 N or from 130 to 180 N, or from 130 to 170 N or from 130 to 160 N, or from 130 to 150 N or from 130 to 140 N, or from 140 to 200 N, or from 140 to 190 N or from 140 to 180 N, or from 140 to 170 N or from 140 to 160 N, or from 140 to 150 N or from 150 to 200 N, or from 150 to 190 N or from 150 to 180 N, or from 150 to 170 N or from 150 to 160 N, or from 170 to 200 N or from 170 to 200 N, or from 170 to 190 N or from 170 to 180 N, or from 180 to 200 N or from 180 to 190 N, or from 190 to 200 N.

The ratio of the triggering force to the loading force may be from about 1:100 or from about 1:90 or from about 1:80 or from about 1:70 or from about 1:60 or from about 1:50 or from about 1:40 or from about 1:30 or from about 1:20 or from about 1:10 or from about 1:5. The ratio of the triggering force to the loading force may be from about 1:100 to about 1:5 or from about 1:90 to about 1:5 or from about 1:80 to about 1:5 or from about 1:70 to about 1:5 or from about 1:60 to about 1:5 or from about 1:50 to about 1:5 or from about 1:40 to about 1:5 or from about 1:30 to about 1:5 or from about 1:20 to about 1:5 or from about 1:10 to about 1:5 or from about 1:100 to about 1:10 or from about 1:90 to about 1:10 or from about 1:80 to about 1:10 or from about 1:70 to about 1:10 or from about 1:60 to about 1:10 or from about 1:50 to about 1:10 or from about 1:40 to about 1:10 or from about 1:30 to about 1:10 or from about 1:20 to about 1:10.

The domes of the present invention have two states, loaded and unloaded. This intermediate energetic state cannot be captured for a non-encased device as the state is highly transient due to the dynamics of snapping-through which makes the dome pass through this state and reach instead the lower energetic state of the fully inverted dome.

The devices of the present invention are capable of being stored for long periods of time without the dome transitioning from the unloaded state to the loaded state. The devices of the present invention may be stored without transitioning from the unloaded state to the loaded state for at least about 6 months or about 1 year or about 2 years or about 3 years or about 4 years or about 5 years or about 6 years or about 7 years or about 8 years or about 9 years or about 10 years or more. The devices of the present invention may be stored without transitioning from the unloaded state to the loaded state for about 1 year to 20 years or from 1 year to 15 years or from 1 year to 10 years or from 1 year to 5 years or from 2 years to 20 years or from 2 years to 15 years or from 2 year to 10 years or from 2 year to 5 years or from 3 years to 20 years or from 3 year to 15 years or from 3 year to 10 years or from 3 year to 5 years or from 4 years to 20 years or from 4 year to 15 years or from 4 years to 10 years or from 4 year to 5 years or from 5 years to 20 years or from 5 years to 15 years or from 5 years to 10 years or from 10 years to 20 years or from 15 years to 20 years.

In applicators for the use in projecting microprojection arrays into skin, the inner environment should be kept dry, and the device stored in ambient or refrigerated conditions, protected from light.

In one embodiment the primed dome is held is in place in the housing and/or applicator by a dome retention ring. In one embodiment the dome retention ring maintains the dome in place within the main body.

In some embodiment of the devices of the present invention the hardness of the steel used for the dome is from about 500 to about 650 HV (Vickers Hardness) pre-heat treatment. The hardness of the steel used for the dome may be from about 400 to about 750 HV or from about 450 to about 750 HV or from about 500 to about 750 HV or from about 550 to about 750 HV or from about 600 to about 750 HV or from about 650 to about 750 HV or from about 700 to about 750 HV or from about 400 to about 700 HV or from about 450 to about 700 HV or from about 500 to about 700 HV or from about 550 to about 700 HV or from about 600 to about 700 HV or from about 650 to about 700 HV or from about 400 to about 650 HV or from about 450 to about 650 HV or from about 500 to about 650 HV or from about 550 to about 650 HV or from about 550 to about 600 HV or from about 600 to about 750 HV or from about 600 to about 700 HV or from about 600 to about 650 HV or from about 540 to about 600 HV.

In some embodiments of the microprojection array applicators and methods of applying the microprojection arrays to the skin the parameters for delivering the microprojection array may be but are not limited to: application energy 65-165 mJ; application energy per projection 40-120 μJ; dome mass 1.5-2.0 g; patch velocity 15-28 m-s-1. In some embodiments of the microprojection array applicators and methods of applying the microprojection arrays to the skin the parameters for the patch may include patch mass 300-600 mg; patch number of projections 1,000-3,000; tip radius 10-100 μm; patch size diameter of 7-20 mm; length of projection 200-800 μm; base width 90-150 μm; projection spacing 100-300 μm. In some embodiments the spacing among the microprojections is equidistant. In one embodiment the mass of the MAP is 600 mg, velocity 20 m/s; length of microprojection from 500-600 μm and the pitch from 190-230.

The speed of the microprojection array as it is projected into the skin depends at least in part upon the area of the array. The range of speeds for the microprojection array entering the skin may be from about 10 m/s to about 50 m/s or from about 10 m/s to about 40 m/s or from about 10 m/s to about 30 m/s or from about 10 m/s to about 25 m/s or from about 10 m/s to about 20 m/s or from about 20 m/s to about 50 m/s or from about 20 m/s to about 40 m/s or from about 20 m/s to about 30 m/s or from about 25 m/s to about 50 m/s or from about 25 m/s to about 40 m/s or from about 25 m/s to about 30 m/s. In preferred embodiments of the microprojection applicators of the present invention the speed of the microprojection array is at least 15 m/s or at least 20 m/s or at least 25 m/s or at least 30 m/s. In some embodiments the velocity of the MAP is from 18 to 28 m/s or from 24 to 28 m/s or from 18-22 m/s.

The microprojection arrays that the applicator of the present invention projects into the skin may have a variety of shapes and sizes. The microprojection array may be square, circular, rectangular or irregular depending on its use. The microprojection arrays can be varied in size depending on its use. The area of the patch will have an impact on the ability to penetrate the subject, but this must be balanced by requirements, including but not limited to the number of projections required to carry sufficient vaccine dosage, skin penetration depth and amount of vaccine delivered into the specific skin window.

The projections are typically separated by between 100 μm and 300 μm, between 100 μm and 250 μm, between 100 μm and 200 μm, between 100 μm and 200 μm, and more typically between 190 μm and 230 μm, leading to patches having between 1000 and 10000 projections per MAP and more typically between 1000 and 3000 projections per MAP. In some embodiments of the microprojection array the number of microprojections is between 1000 and 2500 and in certain embodiments the number of microprojections is 1672, 1992 or 2340.

The length of the projections may be from 100 μm to 1000 μm or from 100 μm to 900 μm or from 100 μm to 800 μm or from 100 μm to 700 μm or from 100 μm to 600 μm or from 100 μm to 500 μm or from 100 μm to 400 μm or from 100 μm to 300 μm or from 100 μm to 250 μm or from 100 μm to 200 μm or from 150 μm to 700 μm or from 150 μm to 600 μm or from 150 μm to 500 μm or from 150 μm to 400 μm or from 150 μm to 300 μm or from 150 μm to 250 μm or from 150 μm to 200 μm or from 200 μm to 700 μm or from 200 μm to 600 μm or from 200 μm to 500 μm or from 200 μm to 400 μm or from 200 μm to 300 μm or from 200 μm to 250 μm or from 225 μm to 700 μm or from 225 μm to 600 μm or from 225 μm to 500 μm or from 225 μm to 400 μm or from 225 μm to 300 μm or from 225 μm to 250 μm or from 250 μm to 700 μm or from 250 μm to 600 μm or from 250 μm to 500 μm or from 250 μm to 400 μm or from 250 μm to 300 μm. The projections may consist of a multiple layer design having one or two or three layer or more layers. The microprojections may have a step shoulder. It may be desirable to incorporate a discontinuity into the effective profile of the projections and this can have benefits either in combination with the convex effective profile, or in isolation. Accordingly, FIG. 20 shows one embodiment of a projection having a stepped effective profile parameters which may be controlled to obtain a desired penetration performance or to provide sufficient material to induce a desired response within the subject. In this embodiment there are three sections to the microprojection: base, middle and tip. As shown in FIG. 20 the base is 300 μm long and tapers along its length from 130 μm to 110 μm. The middle is 152 μm long and tapers from 60 μm to 40 μm. The tip is 100 μm in length and tapers from 20 μm to 10 μm at the top of the microprojection.

The step can assist in ensuring more consistent depth of penetration in different biological subjects, despite variations in the tissue properties from subject to subject. In particular, during insertion of the projection, the step can impact on the dermal tissues, which typically present an increased resistance to penetration compared to tissues in outer layers of the skin (such as the viable epidermis, for example), thereby limiting further penetration of the projection. By providing projections with a suitably configured stepped effective profile, and using controlled application parameters, it is therefore possible to ensure that the tips of the projections extend into the dermis by a predetermined distance.

An advantage of using a stepped effective profile as discussed above is that the support section may be configured to effectively provide mechanical reinforcement for the projection, without impacting on the effective profile of the penetrating end section. This mechanical reinforcement may be provided by merely increasing the diameter of the projections along desired portions of the projection, but may also be provided in other ways, such as by providing buttress features radiating from the base of the projections, to even further strengthen the projections.

The microprojection array may be made of any suitable materials including but not including liquid crystal polymers and plastic. The overall mass of some embodiments of the microprojection array is about 0.3 to 0.6 grams or 0.5 to 0.7 grams. The microprojection array may have bevelled edges to reduce peak stresses on the edge of the array. The microprojection array may have a mass of less than 1.0 grams, or less than 0.9 grams or less than 0.8 grams or less than 0.7 grams, or less than 0.6 grams or less than 0.5 grams or less than 0.6 grams, or less than 0.5 grams or less than 0.4 grams or less than 0.3 grams or less than 0.2 grams or less than 0.1 grams or less than 0.05 grams. The microprojection array may have a mass of about 0.05 grams to about 2 grams, or from about 0.05 grams to about 1.5 grams or from about 0.05 grams to about 1.0 grams or from about 0.05 grams to about 0.9 grams, or from about 0.05 grams to about 0.8 grams or from about 0.05 grams to about 0.7 grams, or from about 0.05 grams to about 0.6 grams or from about 0.05 grams to about 0.5 grams or from about 0.05 grams to about 0.4 grams, or from about 0.05 grams to about 0.3 grams or from about 0.05 grams to about 0.2 grams, or from about 0.05 grams to about 0.1 grams or from about 0.1 grams to about 1.0 grams or from about 0.1 grams to about 0.9 grams, or from about 0.1 grams to about 0.8 grams or from about 0.1 grams to about 0.7 grams, or from about 0.1 grams to about 0.6 grams or from about 0.1 grams to about 0.5 grams or from about 0.1 grams to about 0.4 grams, or from about 0.1 grams to about 0.3 grams or from about 0.1 grams to about 0.2 grams. In one embodiment of the applicator/microprojection system the mass of the array is about 0.3 grams, the array is projected at a velocity of about 20-26 m/s by the applicator.

The present invention relates to microprojection array applicators that provide application of microprojection arrays to the skin for the delivery of substances in particular the delivery of vaccine antigens. The present invention also relates to methods of using the microprojection array applicators for applying microprojection arrays to the skin of a subject. The applicators and methods of the present invention are especially useful for the delivery of high density microprojection arrays to the skin surface. The applicators and methods of the present invention are also useful for the delivery of high density microprojection arrays at a high rate of speed to the skin surface. The present invention is designed to achieve tolerable penetration for high density, low mass microprojection arrays (>1,000/cm²) that are delivered to the skin at high velocities.

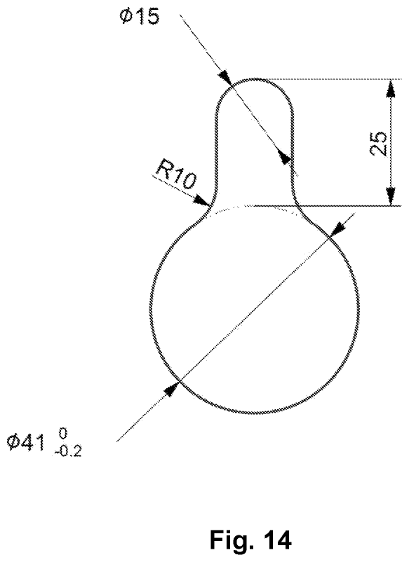

The bottom of the microprojection array applicator is covered with a foil sheet to keep the device sterile. A schematic drawing with dimension of one embodiment of the foil seal is shown in FIG. 14. The foil can be an aluminum foil and other layers of materials such as but not limited to polyethylene layers may be added to the aluminum layer. In some embodiments such as that shown in FIG. 14 the foil seal may have a tab as part of the seal which can be used to remove the foil seal from the device prior to administration. The can may have a collapsible section which acts as a trigger to activate the dome. The collapsible section or sections of the can may be on upper section of the device. Preferably the flexible or collapsible section of the can is actuated through a force applied by hand such that application of the microprojection array is comfortable to both the patient and the person activating the applicator. In one embodiment of the applicator of the present invention the force is applied to the applicator in a fashion that is substantially perpendicular to the skin to which the microprojection array is applied such that the force travels down through the dome. Alternatively, the activation force could be applied in a direction substantially parallel to the skin by a mechanism that may be actuated between the thumb and forefinger. The mechanism by which the applicator is activated should not cause discomfort to the patient.

The microprojection array may be propelled from the device after the device is activated such that the microprojection array transits a distance between the applicator device and the target skin and then penetrates the skin. In essence, the microprojection array may be propelled across some distance and then penetrate the target skin. In one embodiment of the applicator where the microprojection array is discharged from the device, the microprojection array could be tethered to a mechanism that protrudes through the dome such that when the dome is activated the mechanism releases the microprojection array with sufficient force to propel the array into the skin. The spigot provides attachment of the MAP to the main body. The spigot enables guided travel of the microprojection array to ensure that the microprojection array contacts the skin. This should permit a high speed, low mass, pain free delivery of the microprojection array to the skin. In another embodiment the microprojection array may be attached to a low mass tether. In this embodiment the microprojection array is either not in direct contact with the dome or the only contact between the cantilevered ring and the microprojection array is when the dome impacts the array sending the array toward the skin. In these cases, the microprojection array can be struck at the point where the dome achieves maximum velocity and the mass of the cantilevered ring does not impact the skin of the patient. In preferred embodiments of the applicator device of the present invention the microprojection array is either propelled without attachment to the device or attached to the device via a low mass connector such as a tether. In an alternative embodiment the patch insertion and flight guiding may be accomplished with springs instead of a sliding spigot.

A desiccant may be included in the microprojection array applicator to create a dry internal environment and reduce water ingress. One method of incorporating a desiccant into the applicator is by incorporating the desiccant into the foil seal or as a component of the main body. A desiccant ring or a solid or semi-solid desiccant can be placed within the main body in a discrete section of the main body to provide a dry internal environment.

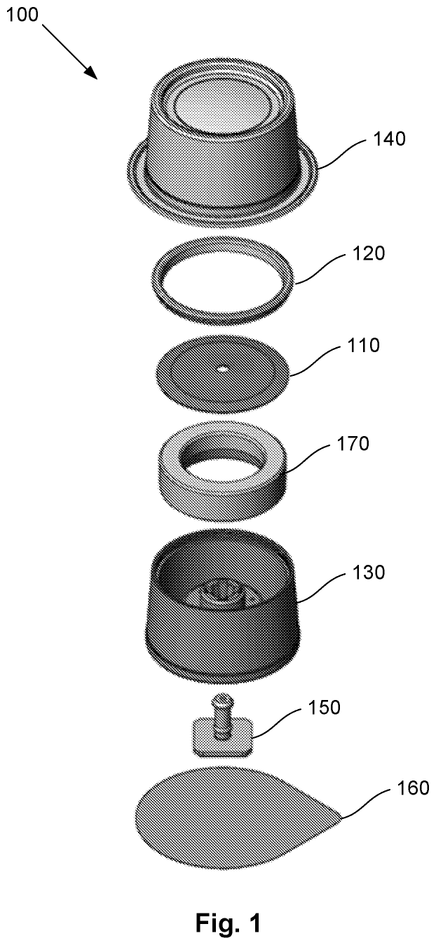

In a preferred embodiment of the device of the present invention the device 100 comprises six components as shown in FIG. 1: a dome 110; a MAP 150; a can 140; a main body 130; a retaining ring 120 and a foil seal 160. In addition, another component that entrains a desiccant 170 can optionally be added to the device. Alternatively, the desiccant may be entrained in the other plastic components of the device or free desiccant may be added into the device.

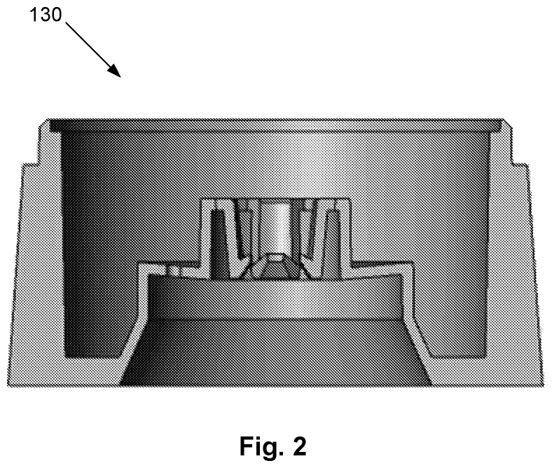

The main body 130 of the device is a molded polymer component as seen for example in FIG. 2. The molded polymer insert may be made of varying polymer materials preferably high-density polyethylene (HDPE) which can withstand high impact, possess high-stress crack resistance, and have extreme durability. HDPE is preferred for the main body 130 because the laminate on the foil needs to be PE for gamma compatibility. The main body 130 is shaped into a cylinder in which there is an opening at the bottom of the cylinder. The walls of the cylinder are slightly angled such that the top of the main body is larger in diameter than the bottom of the cylinder. In one embodiment the top of the main body 130 has a diameter of 33.2 mm and the bottom opening has a diameter of 40 mm. Within the main body 130 are molded several features. The top portion of the main body 130 may have several features by which the dome 110 and the retaining ring 120 are fitted to the main body 130. The dome 110 may be seated onto a ledge that has been molded into the top. The ledge is approximately 0-20 mm or 0-10 mm from the top of the main body 130 and is approximately 3-5 mm in width. The main body 130 may have several feature including having ridges or flutes in the walls of the main body 130, that could be used to assist with heat sealing process which fuses the can to the main body 130.

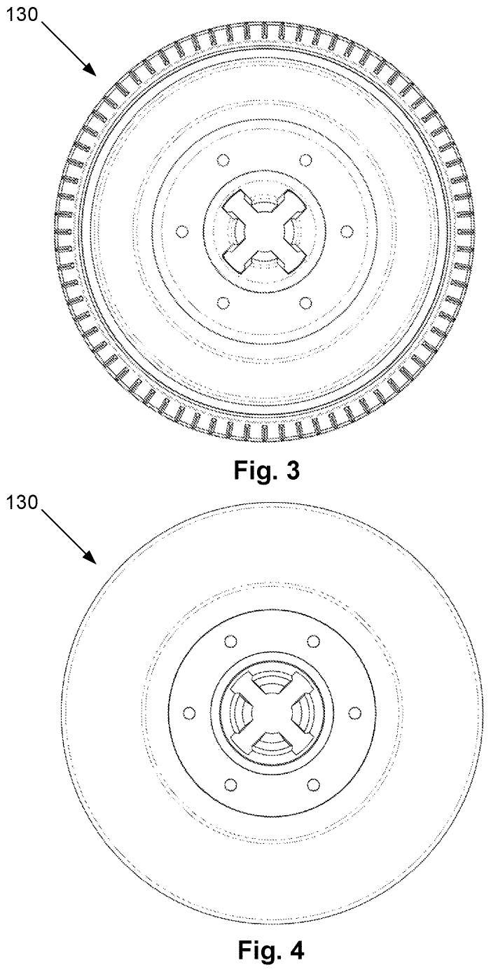

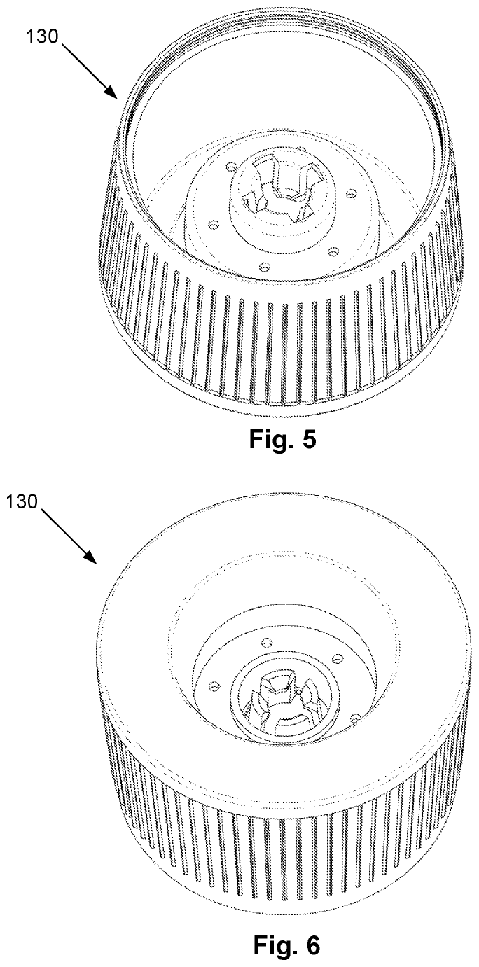

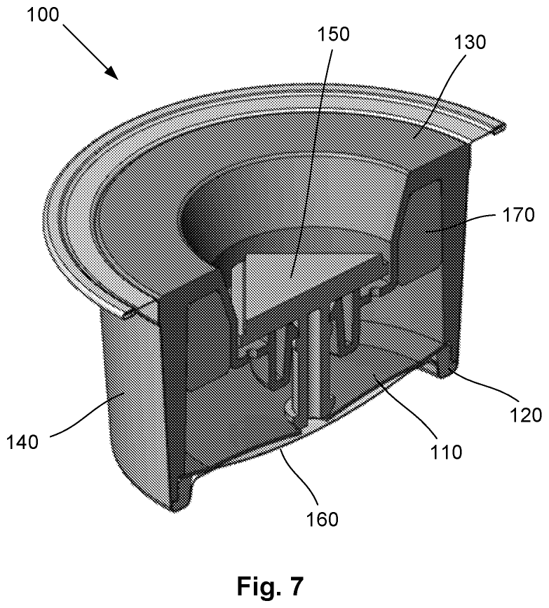

In one embodiment the height of the main body 130 is approximately 23.0 mm. The main body 130 is molded in such a way as to provide a flat surface at its base with a void at the center of the base which is approximately 22 mm in diameter. The flat surface of the main body 130 is approximately 36 mm in diameter. This void will permit the skin of the subject to raise up into the void. The main body 130 is molded such that the flat base extends into the inner portion of the main body 130 such that the main body 130 provides an attachment site for the MAP 150. Referring to FIGS. 3-7 the molded material in the inner portion of the main body 130 may be formed into a ring within which another ring is formed to have one or more sites for attaching the MAP 150 to the main body 130. These attachments sites can take the form of extended arms at which the terminus has a portion which can interact with the spigot of the MAP 150 to lock the MAP 150 in place in the main body 130 until the dome 110 is activated to force the MAP 150 out of position. The locking mechanism may be a continuous ring or comprise one more arms, including two arms or three arms or four arms. One embodiment of the attachment of the MAP 150 to the main body in shown in FIG. 7.

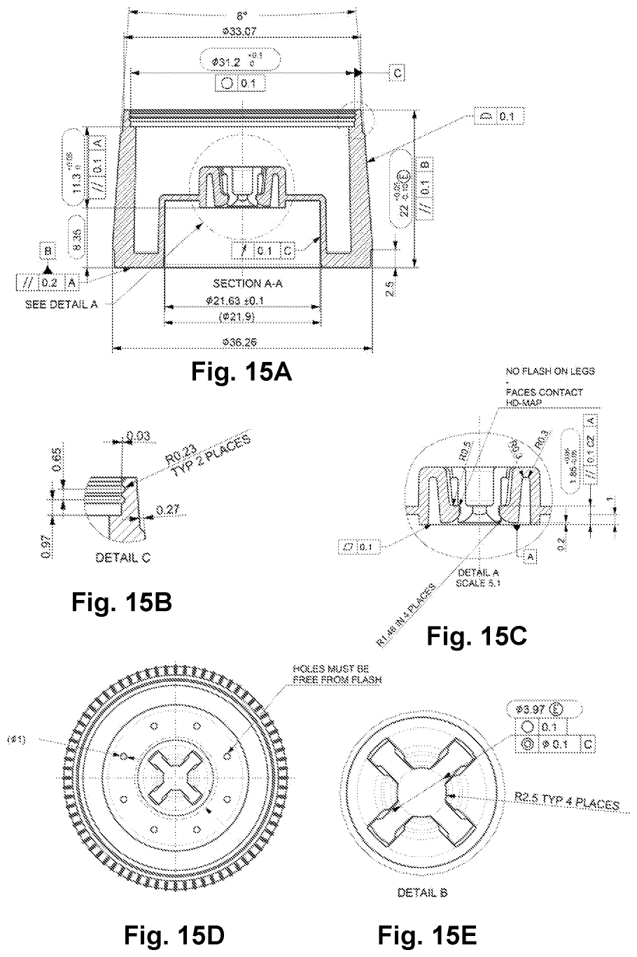

FIG. 15A is a cross-sectional schematic diagram including the dimensions of one embodiment of the main body of the device. This figure includes the retaining ring but does not show the dome spring. FIG. 15B is a magnified cross-sectional schematic diagram of the interface between the main body and the retaining ring of the embodiment shown in FIG. 15A. The dimensions of the ribs of the retaining ring and the indentations of the main boy are shown. In this embodiment the retaining ring has two ribs or curved rims that interface with indentation in the top of the main body such that the retaining ring remains in place attached to the main body.

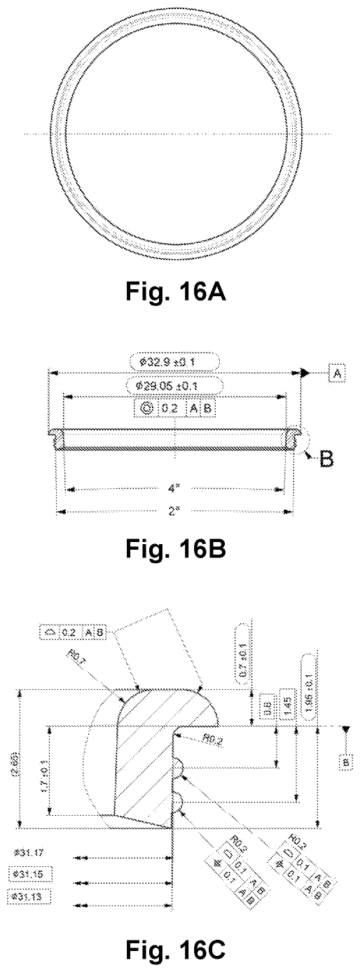

FIG. 16A is a schematic drawing of the top of the retaining ring; FIG. 16B is a schematic cross-sectional drawing of the retaining ring; and FIG. 16C is a magnified portion of the schematic drawing in 16B that shows the details of the exterior surface of the retaining ring. These diagrams show the dimensions of one embodiment of the retaining ring. FIG. 15C is a magnified cross-sectional schematic diagram of the portion of the main body which holds the spigot/MAP in place until the device is activated. The figure shows how the spigot is held in place until use. FIG. 15D is a schematic diagram of the top view of the main body and FIG. 15E is an exploded view of the center of the main body.

As previously described, the main body 130 and thus the device 100 has a void in the bottom which permits the MAP 150 to be projected from its attachment to the main body 130 into the skin of the subject. In one embodiment of the present invention the potential travel distance of the MAP 150 to the skin is about 5 to 7 mm. This distance may vary depending on the characteristics of the skin of the subject as the device 100 is placed on the skin of the subject with slight pressure such that the skin of the subject forms a bubble in the void of the device 100. The potential travel distance of the MAP 150 from its fixed position in the main body 130 of the device 100 to the point where it is retained by the device 100 is approximately 8 to 10 mm.

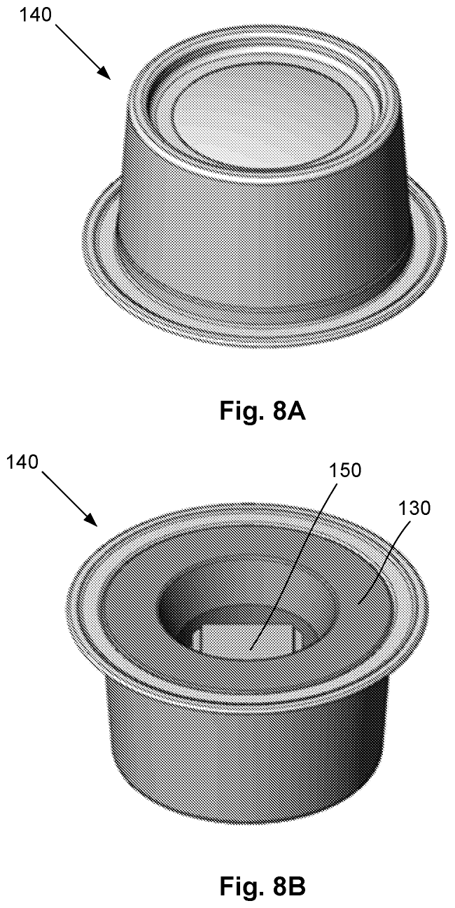

The can 140 which is the exterior portion of the applicator device 100 is made of aluminium over which can be layered printing, a protective lacquer and a polymer laminate. One embodiment of the can 140 is shown in FIGS. 8A and 8B. The thickness of the can 140 is approximately 90-180 μm or from 90-120 μm. As can be seen in from the figure the can 140 may be tapered from the top to the bottom such that the diameter of the can 140 at the top is approximately 31.6 mm and the diameter at the bottom is approximately 44.85 mm. The bottom of the can 140 has a flange such that when the main body 130 is inserted into the can 140 the can 140 and the main body 130 may be joined by heat pressing the main body 130 into the can 140.

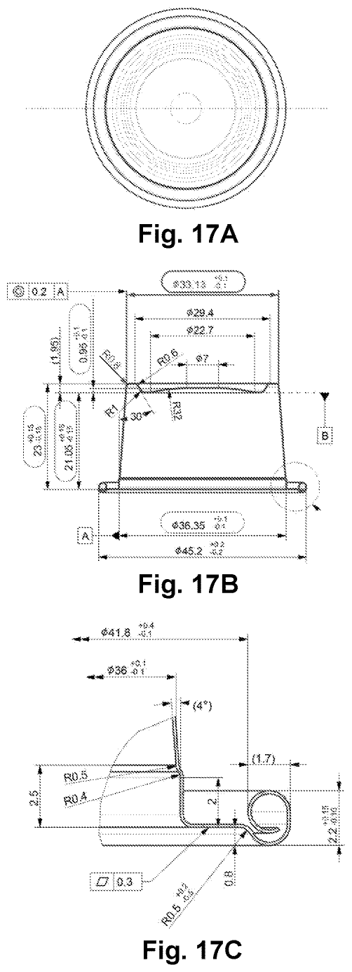

FIG. 17A is a top view of a schematic drawing of the can 140 exterior covering for the device 100. FIG. 17B is a cross-sectional schematic drawing of one embodiment of the can 140 covering with dimensions. FIG. 17C is an enlarged cross sectional view of the interface of the can 140 covering with the main body 130.

The retaining ring 120 is superior to prior methods of entraining the dome 110 in the main body 130 in that there is improved consistency of the transfer of energy from the dome 110 to the MAP 150. The use of a retaining ring 120 also simplifies manufacture by removing the need for ultrasonic welding.

The bottom of the device 100 has a closure system, such as a foil seal 160 that may be opened or removed before application of the microprojection array.

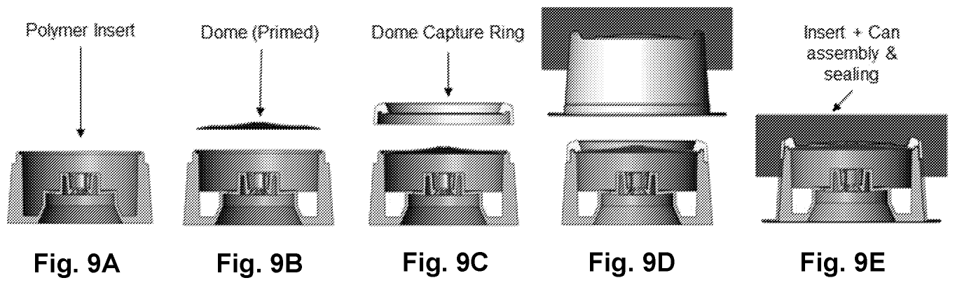

The device may be constructed by following steps as illustrated in FIGS. 9A to 9E. The main body is provided as shown in FIG. 9A and a desiccant may be optionally fit into the main body at this stage. The next step as shown in FIG. 9B involves locating the primed dome onto the seat within the main body of the device. Next, as shown in FIG. 9C, the retaining ring is press fit to the main body securing the dome in place. The main body is then inserted into the can as shown in FIG. 9D, and then heat pressed so that the main body is adhered to the can as shown in FIG. 9E. The MAP is then inserted into the main body such that the retention features of the main body hold the MAP in place. Finally, the foil seal is heat pressed to the base of the can.

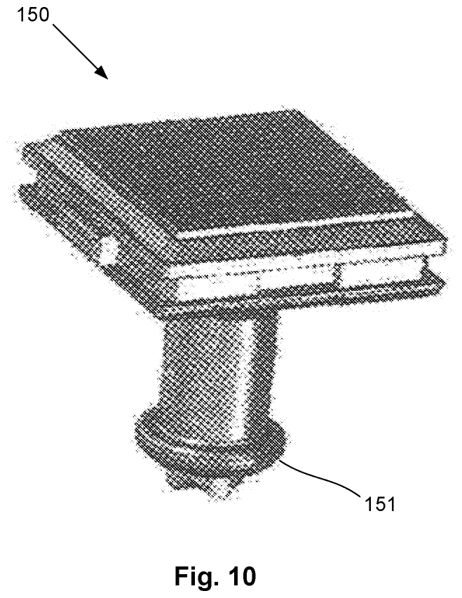

In another embodiment the back of the microprojection array has a detail that allows it to engage with the device. For example, this could be in the form of a spigot, magnet or other shape that mechanically allows the back of the microprojection array to connect to the firing mechanism. In a preferred embodiment of the MAP 150, the detail is a spigot as shown in FIG. 10. This detail, be it a bore or a spigot is further used as a guide to ensure that the patch during flight tracks perpendicular to the device and cannot strike the skin at an inappropriate angle. The detail on the back of the microprojection array also tethers the patch to the device, in this way the patch is free to “fly” forward and strike the skin but is still attached to the device, this renders the device safe as the patch cannot be shot out of the device and allows the patch and the applicator to be removed from the skin as a single unit. The detail on the back of the patch allows the patch to be fired forward onto the skin, this forward movement is restricted in that the patch is still connected to the applicator and cannot come all the way out. In this way when the applicator is removed from the skin the patch comes away with it. The detail on the back can also allow the patch to be engaged in the device at various positions. In a preferred embodiment the spigot is molded with the MAP 150 as a single unit.

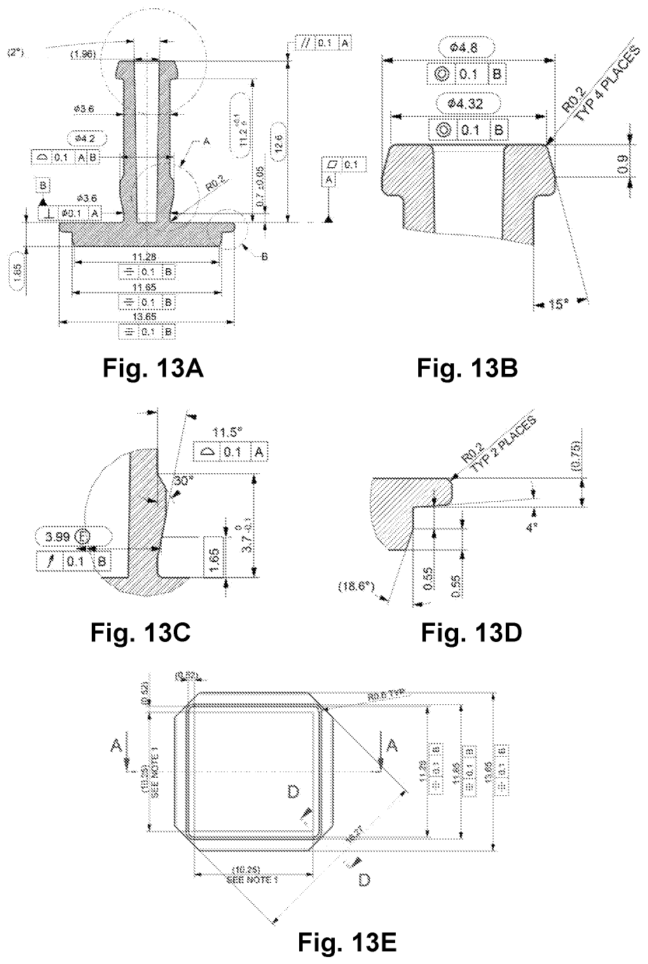

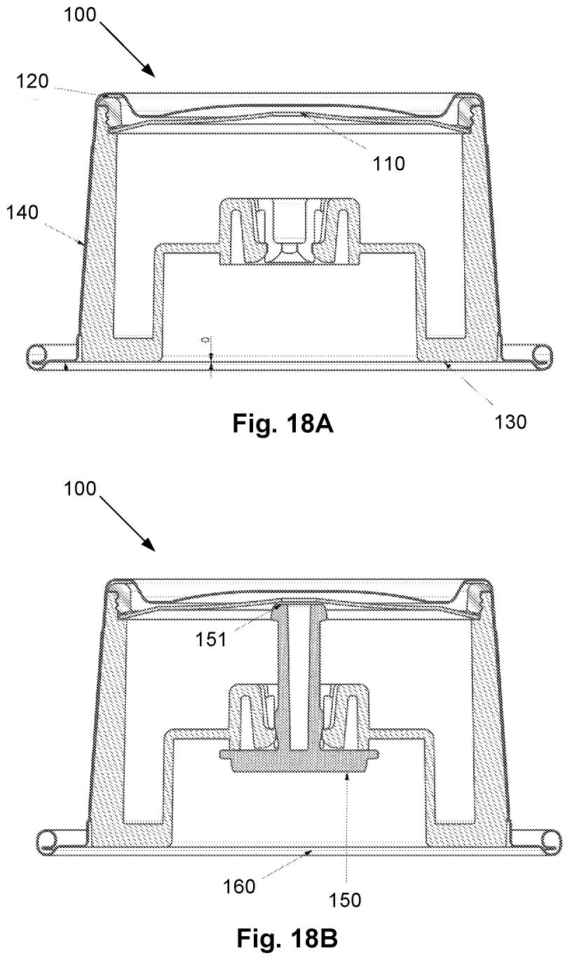

FIG. 13A is a cross-sectional drawing of one embodiment of the MAP with the spigot as a molded unit which shows the dimensions of the molded unit. FIG. 13B is a detailed cross-sectional drawing of the top portion of the MAP unit shown in FIG. 13A. The figure includes the dimensions of one embodiment of this portion of the unit. This top portion of the spigot interfaces with the dome spring such that when the dome spring is activated it contacts the top of the spigot thereby pushing the spigot and MAP forward towards the skin. FIG. 13C is a detailed cross-sectional drawing of the portion of the MAP which is just above the top surface of the MAP shown in FIG. 13A. The figure includes the dimensions of one embodiment of this portion of the unit. The bulged out portion of the spigot interfaces with the main body holding the spigot/MAP unit in place until the dome spring is activated. Once the dome spring is activated and hits the top of the spigot the force is sufficient to release the MAP from the main body by forcing these bulged areas of the spigot to push past the catches in the main body (See FIG. 18B). FIG. 13D is a detailed cross-sectional drawing of the edge portion of the MAP shown in FIG. 13A. The figure include the dimensions of one embodiment of this portion of the unit. As the figure demonstrates there is an edge around the periphery of the MAP. FIG. 13E is a schematic drawing of a top view of the MAP. The figure include the dimensions of one embodiment of the MAP. The top surface of the MAP is attached to the spigot and the bottom surface of the MAP contains the microprojections that enter the skin. This embodiment of the MAP is square, but the shape of the MAP can be rectangular, circular, triangular or any shape that facilitates its loading with active pharmaceuticals ingredients including vaccines.

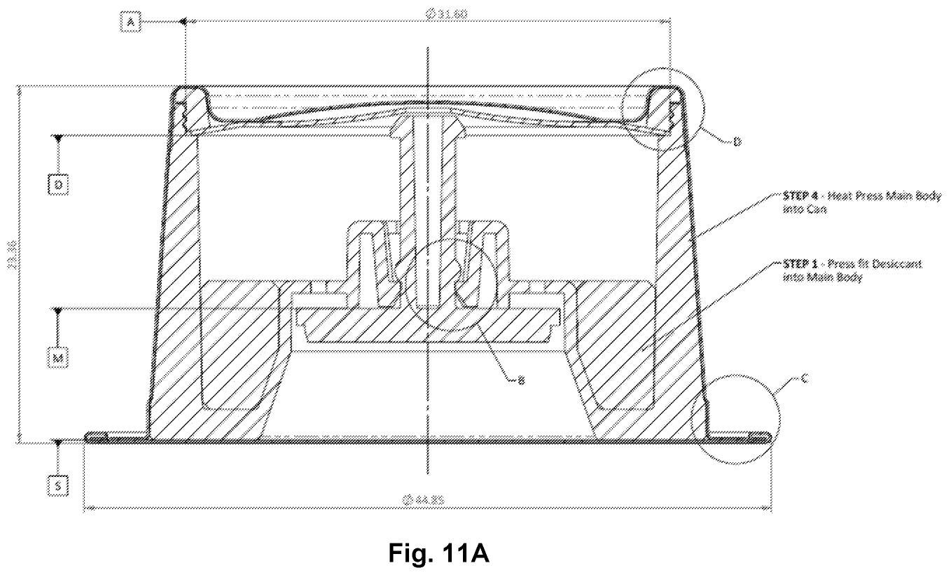

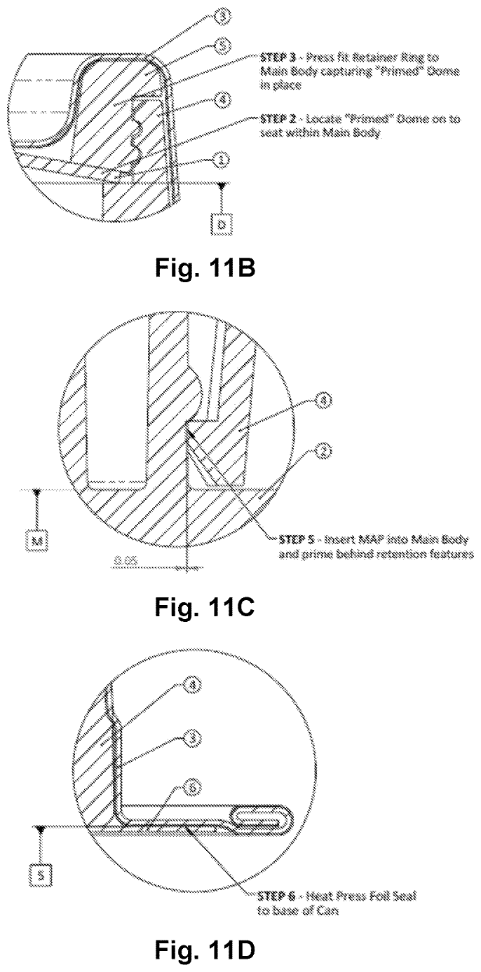

FIG. 11A-D are schematic cross-sectional drawings of one embodiment of the assembled device which highlight the attachment of the dome and retaining ring to the main body, the attachment of the can to the main body and foil seal, and the insertion of the MAP with the spigot into the main body. The applicator device and methods of manufacturing the device and the methods of using the device provide a one-piece shell so that there is no necking or sealing process required making the manufacture easier and more efficient. The device is also amenable to the coating of the MAPs in trays and for the assembly of the coated MAPs into a pre-assembled sterile applicator.

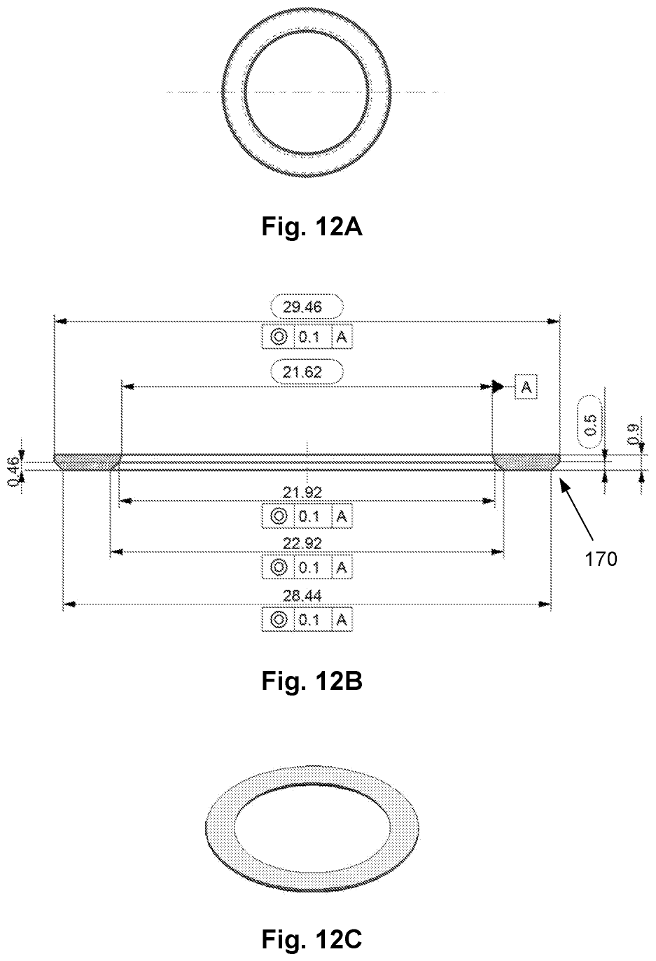

FIG. 12 is a schematic cross-sectional drawing of one embodiment of the desiccant ring which can be placed inside the can and rest upon the main body.

FIG. 18A a schematic cross-sectional drawing of one embodiment of the device 100 which contains the main body 130, retaining ring 120, dome 110 spring, can 140 and foil seal but does not show the spigot/MAP 150. FIG. 18B a schematic cross-sectional drawing of one embodiment of the device 100 which contains the main body 130, retaining ring 120, dome 110 spring, can 140, foil seal 160, and the spigot/MAP 150.

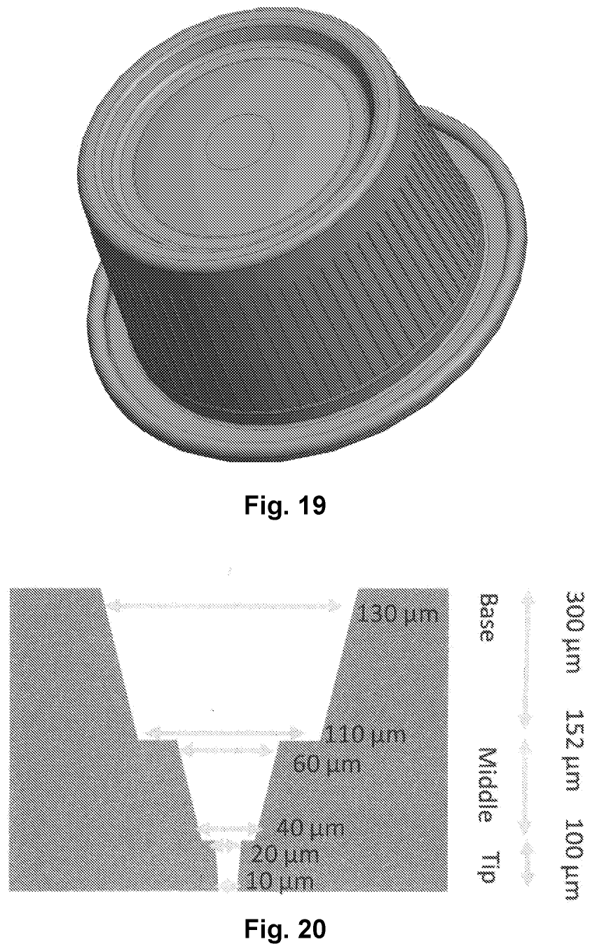

FIG. 19 shows an angled view of the exterior of one embodiment of the applicator device.

The microprojections of the microprojection array can be coated with fluids including pharmaceuticals and biological materials which are deposited onto microprojection arrays providing improved efficiency and precise coating of three dimensional substrates. Print head devices may provide simultaneous two dimension deposition of pharmacological grade biological material in an aseptic environment. These printing devices provide for the coating of different antigens on different microprojections of a microprojection array. These devices can also deposit different antigens and different adjuvants or excipients on any microprojection on a microprojection array. Such biological fluids include vaccines and biopharmaceuticals which pose an additional challenge for coating in that the active material may only be available in low concentrations such as 1-10 mg/mL. This in turn may require that multiple administrations of the material with drying time in between administrations of the material on each feature in order to achieve the targeted therapeutic dose. As the total fluid delivered may be relatively high (the number of drops) it is important to efficiently deposit the material so that the total time to coat the substrate is not excessive.

The microprojections of the microprojection array may be coated with vaccine antigen formulations. The antigens may be derived from pathogenic organisms which include, but are not limited to, viruses, bacteria, fungi parasites, algae and protozoa and amoebae. The vaccine antigens can be protein, peptides, nucleic acids, carbohydrates any material that will provoke an immune response. The microprojection arrays may be coated with cancer vaccines.

Within this disclosure, any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While this invention has been particularly shown and described with references to example embodiments thereof, it will be understood by those skilled in the art that the various changes in form and details may be made therein without departing from the scope of the invention encompassed by the appended claims.

Further advantages of the present immunological compositions and adjuvants of the present invention can be achieved by those skilled in the art based upon the embodiments described herein and are thus specifically within the scope of the present invention.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

It will of course be realised that whilst the above has been given by way of an illustrative example of this invention, all such and other modifications and variations hereto, as would be apparent to persons skilled in the art, are deemed to fall within the broad scope and ambit of this invention as is herein set forth.

1. A device for delivering a microprojection array into the skin of a mammal comprising:
a primed dome;
a retaining ring;
a main body;
a can;
a microprojection array; and
a foil seal.

2. The device of claim 1, wherein the device is a self-contained unit, and wherein the components are contained within the can and the device has no necking or sealing on the exterior of the can.

3. The device of claim 1, wherein the microprojection array comprises a base having a plurality of microprojections and a spigot molded to the base, and wherein the spigot attaches to the main body.

4. The device of claim 1, wherein the dome has a flattened outer edge.

5. The device of claim 4, wherein the flattened outer edge is from 3.3 to 3.6 mm.

6. The device of claim 4, wherein the retainer ring continuously presses on the flattened outer edge of the dome.

7. The device of claim 1, wherein the can is made of aluminum.

8. The device of claim 7, wherein the aluminum is from 90 to 180 μm thick.

9. The device of claim 7, wherein the aluminum is from 90 to 120 μm thick.

10. The device of claim 1, further comprising a desiccant.

11. The device of claim 10, wherein the desiccant is included inside the device.

12. The device of claim 11, wherein the desiccant is included in the foil seal.

13. The device of claim 11, wherein the desiccant is contained within the main body.

14. The device of claim 11, wherein the desiccant is contained within a desiccant ring.

15. The device of claim 1, wherein the microprojection array has from about 1000 to 3000 microprojections.

16. A device for delivering a microprojection array into the skin of a mammal comprising a primed dome seated within a main body which is held in place by a retaining ring, wherein the microprojection array is held by the main body and the main body is contained within a can which has a single opening, and wherein a foil seal is attached to the can opening.

17. A method for assembling a device for delivering a microprojection array into the skin of a mammal comprising:
locating a primed dome onto a seat within a main body of the device;
press fitting a retaining ring to the main body of the device thereby securing the dome in place;
inserting the main body of the device into a can and heat pressing the can so that the main body is adhered to the can;
inserting the microprojection array into the main body such that one or more retention features of the main body hold the microprojection array in place; and
heat pressing a foil seal to the base of the can.

18. (canceled)

19. The method of claim 17, further comprising placing a desiccant ring onto the main body prior to press fitting the retaining ring to the main body.
